(19) United States
(12) Patent Application Publication
(10) Pub. No.: US 2007/0265595 A1
(43) Pub. Date: Nov. 15, 2007

(54) Treatment tool inserting/withdrawing auxiliary device and medical procedure through endoscope

(75) Inventors: Satoshi Miyamoto, Tokyo (JP); Kousuke Motai, Tokyo (JP); Takehiro Nishiie, Tokyo (JP); Junichi Muramatsu, Tokyo (JP); Yasuyuki Kuroda, Hatano-shi (JP); Tadashi Kousai, Fujinomiya-shi (JP); Junichi Kobayashi, Fujinomiya-shi (JP); Yasushi Kinoshita, Fujinomiya-shi (JP)

Correspondence Address: SCULLY SCOTT MURPHY & PRESSER, PC, 400 GARDEN CITY PLAZA, SUITE 300, GARDEN CITY, NY, 11530, US

(73) Assignees: OLYMPUS MEDICAL SYSTEMS CORP., TOKYO (JP); TERUMO KABUSHIKI KAISHA, TOKYO (JP)

(21) Appl. No.: 11/430,739

(22) Filed: May 9, 2006

(51) Int. Cl. A61M 25/00

(52) U.S. Cl. 604/528

(57) ABSTRACT

The invention includes: an operation wire which is inserted into a catheter, and is supported on a distal end or the vicinity of the catheter; and an operation portion which moves the operation wire back and forth with respect to the catheter. The catheter is provided with a curvable portion wherein the distal direction of the catheter is curved with respect to the proximal direction of the catheter.

### BACKGROUND OF THE INVENTION

1. Field of the Invention

The present invention relates to a treatment tool inserting/withdrawing auxiliary device and a medical procedure through an endoscope.

2. Description of Related Art

For performing cannulation (selective insertion into a pancreatic duct/bile duct) in diagnosis and treatment of a pancreatic/bile duct system, while a treatment tool such as a contrast medium injecting catheter is inserted into a channel of a flexible endoscope, back-and-forth moving operation, angling operation, and twisting operation of the endoscope insertion portion, raising-up operation of a forcep stage arranged on the distal end of the insertion portion, and back-and-forth moving operation of the contrast medium injecting catheter with respect to the channel are performed in combination. In this case, it is necessary to match the axial directions of the distal end of the contrast medium injecting catheter and the bile duct (or the pancreatic duct) by delicate operations of the endoscope and the contrast medium injecting catheter. As a result, a high skill for positioning is required for the operator.

Therefore, a contrast medium injecting catheter for facilitating these operations is disclosed in Japanese Unexamined Patent Application, First Publication No. 2002-272675 and Published Japanese Translation No. 2004-532668 of PCT International Publication. Moreover, in a medical procedure other than for the bile duct system, there is an attempt to facilitate the manipulation by means of a catheter as disclosed in U.S. Pat. No. 6,659,981.

According to these, since the distal end of the catheter is curved to some extent, the positioning operation by the endoscope insertion portion can be assisted.

On the other hand, after the cannulation, in order to exchange the contrast medium injecting catheter and a treatment tool required for the subsequent treatment, a guide wire having at least twice the length of the contrast medium injecting catheter is inserted into the contrast medium injecting catheter, and the contrast medium injecting catheter is withdrawn from the endoscope through the guide wire. Then, the treatment tool to be used is inserted into the endoscope along the guide wire, and moved to the target site.

### SUMMARY OF THE INVENTION

An object of the present invention is to provide a device and a method capable of readily performing cannulation while the distal end of an endoscope insertion portion is positioned with respect to the duodenal papilla, and capable of exchanging treatment tools without using a guide wire.

The treatment tool inserting/withdrawing auxiliary device according to a first aspect of the present invention includes: a catheter through which a treatment tool for an endoscope is to be inserted, and which is insertable into a channel of a flexible endoscope; an operation wire which is inserted into the catheter, and is supported on a distal end or the vicinity of the catheter; an operation portion which moves the operation wire back and forth with respect to the catheter; and a curvable portion which is provided on the catheter and is curved by a back and forth movement of the operation wire, wherein the curvable portion is curvable within a range between 0 to 170 degree of a curved angle, assuming that the curved angle is an angle defined by axial centers of the proximal end and the distal end of the curvable portion, and the curved angle becomes 0 when the axial centers of the proximal end and the distal end are approximately on the same line having the curvable portion therebetween.

Moreover, the medical procedure through an endoscope according to the first aspect of the present invention includes: inserting the distal end of the catheter of the treatment tool inserting/withdrawing auxiliary device, the distal direction of which is curved at a predetermined angle with respect to the proximal direction, through the channel of the flexible endoscope, to arrange in the vicinity of the duodenal papilla; matching the distal direction of the catheter with the direction of a bile duct or pancreatic duct, while the distal end of the endoscope is fixed; inserting a first treatment tool into the catheter; and making the first treatment tool project from the catheter, to be inserted into the bile duct or pancreatic duct.

### BRIEF DESCRIPTION OF THE DRAWINGS

FIG. 1 is an overall schematic diagram showing a treatment tool inserting/withdrawing auxiliary device according to a first embodiment.

FIG. 2 is a main part side view showing a curvable catheter of the treatment tool inserting/withdrawing auxiliary device according to the first embodiment.

FIG. 3 is a cross-sectional view taken along the line A-A′ in FIG. 2.

FIG. 4 is a cross-sectional view taken along the line B-B′ in FIG. 2.

FIG. 5 is an internal configuration diagram of the main part showing the curvable catheter of the treatment tool inserting/withdrawing auxiliary device according to the first embodiment.

FIG. 6 is an internal configuration diagram of the main part showing the curvable catheter of the treatment tool inserting/withdrawing auxiliary device according to the first embodiment.

FIG. 7 is an internal configuration diagram of the main part showing a modified example of the curvable catheter of the treatment tool inserting/withdrawing auxiliary device according to the first embodiment.

FIG. 8 is an explanatory diagram showing the curved condition of the curvable catheter of the treatment tool inserting/withdrawing auxiliary device according to the first embodiment.

FIG. 9 is a cross-sectional view showing a branched portion of the treatment tool inserting/withdrawing auxiliary device according to the first embodiment.

FIG. 10 is a cross-sectional view showing a modified example of the branched portion of the treatment tool inserting/withdrawing auxiliary device according to the first embodiment.

FIG. 11 is an explanatory diagram showing a medical procedure through an endoscope by the treatment tool inserting/withdrawing auxiliary device according to the first embodiment.

FIG. 12 is an explanatory diagram showing the medical procedure through the endoscope by the treatment tool inserting/withdrawing auxiliary device according to the first embodiment.

FIG. 13 is an explanatory diagram showing the medical procedure through the endoscope by the treatment tool inserting/withdrawing auxiliary device according to the first embodiment.

FIG. 14 is an explanatory diagram showing the medical procedure through the endoscope by the treatment tool inserting/withdrawing auxiliary device according to the first embodiment.

FIG. 15 is an explanatory diagram showing the medical procedure through the endoscope by the treatment tool inserting/withdrawing auxiliary device according to the first embodiment.

FIG. 16 is an internal configuration diagram of the main part showing the curvable catheter of the treatment tool inserting/withdrawing auxiliary device according to a second embodiment.

FIG. 17 is an explanatory diagram showing a curved condition of the curvable catheter of the treatment tool inserting/withdrawing auxiliary device according to a third embodiment.

FIG. 18 is a main part plan view showing the curvable catheter of the treatment tool inserting/withdrawing auxiliary device according to the third embodiment.

FIG. 19 is an overall schematic diagram showing the treatment tool inserting/withdrawing auxiliary device according to a fourth embodiment.

FIG. 20 is a main part plan view showing the curvable catheter of the treatment tool inserting/withdrawing auxiliary device according to a fifth embodiment.

FIG. 21 is a main part plan view showing a modified example of the curvable catheter of the treatment tool inserting/withdrawing auxiliary device according to the fifth embodiment.

FIG. 22 is an explanatory diagram showing a curved condition of the curvable catheter of the treatment tool inserting/withdrawing auxiliary device according to the fifth embodiment.

FIG. 23 is an explanatory diagram showing a medical procedure through an endoscope by the treatment tool inserting/withdrawing auxiliary device according to the fifth embodiment.

FIG. 24 is an explanatory diagram showing the curved condition of the treatment tool inserting/withdrawing auxiliary device according to the fifth embodiment.

FIG. 25 is an explanatory diagram showing the medical procedure through the endoscope by the treatment tool inserting/withdrawing auxiliary device according to the fifth embodiment.

FIG. 26 is an explanatory diagram showing a curved condition of the curvable catheter of the treatment tool inserting/withdrawing auxiliary device according to a sixth embodiment.

FIG. 27 is a main part plan view showing the curvable catheter of the treatment tool inserting/withdrawing auxiliary device according to a seventh embodiment.

FIG. 28 is a main part cross-sectional view showing the curvable catheter of the treatment tool inserting/withdrawing auxiliary device according to the seventh embodiment.

FIG. 29 is an explanatory diagram showing a medical procedure through an endoscope by a modified example of the treatment tool inserting/withdrawing auxiliary device.

FIG. 30 is an explanatory diagram showing the medical procedure through the endoscope by the modified example of the treatment tool inserting/withdrawing auxiliary device.

FIG. 31 is a main part side view showing the curvable catheter of the modified example of the treatment tool inserting/withdrawing auxiliary device.

FIG. 32 is a main part cross-sectional view showing the curvable catheter of the modified example of the treatment tool inserting/withdrawing auxiliary device.

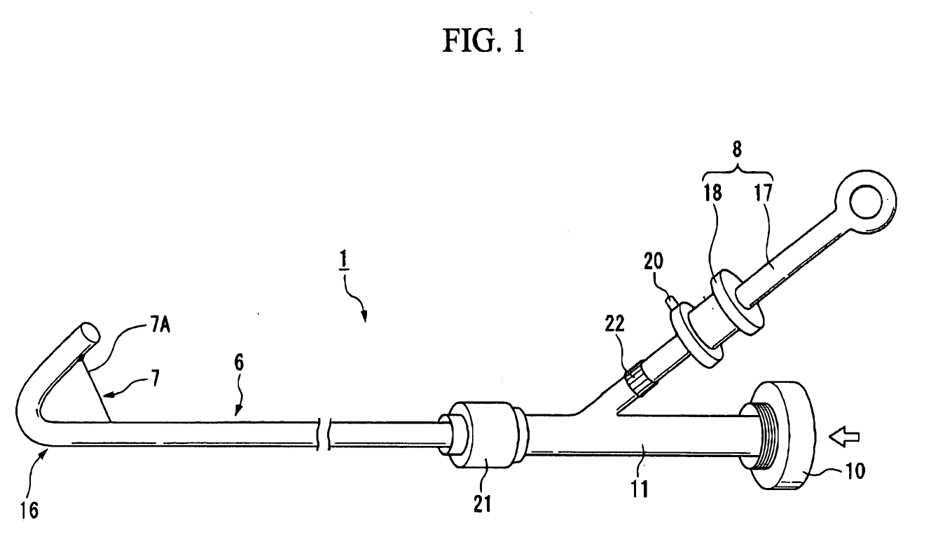

### DESCRIPTION OF THE PREFERRED EMBODIMENTS

Hereunder is a detailed description of preferred embodiments according to the present invention. In the following description, the same reference symbols are used for the same components, and duplicate descriptions are omitted.

### First Embodiment

A treatment tool inserting/withdrawing auxiliary device 1 according to the present embodiment includes: a curvable catheter (catheter) 6 having a through hole 6A through which a contrast medium injecting catheter (treatment tool for an endoscope, first treatment tool) 2 can be inserted, and which can be inserted into a channel 5 of a flexible endoscope 3; an operation wire 7 which is inserted into the curvable catheter 6, and is partially exposed to the outside of the curvable catheter 6 to form an exposed portion 7A supported on a distal end of the curvable catheter 6; an operation portion 8 which moves the operation wire 7 back and forth with respect to the curvable catheter 6; a treatment tool insertion/withdrawal port 10 which is provided in communication with the curvable catheter 6, and through which the contrast medium injecting catheter 2 can be inserted/withdrawn; and a branched portion 11 which is connected to a proximal end of the curvable catheter 6, and has the through hole 6A of the curvable catheter 6 branched into the operation portion 8 side and the treatment tool insertion/withdrawal port 10 side.

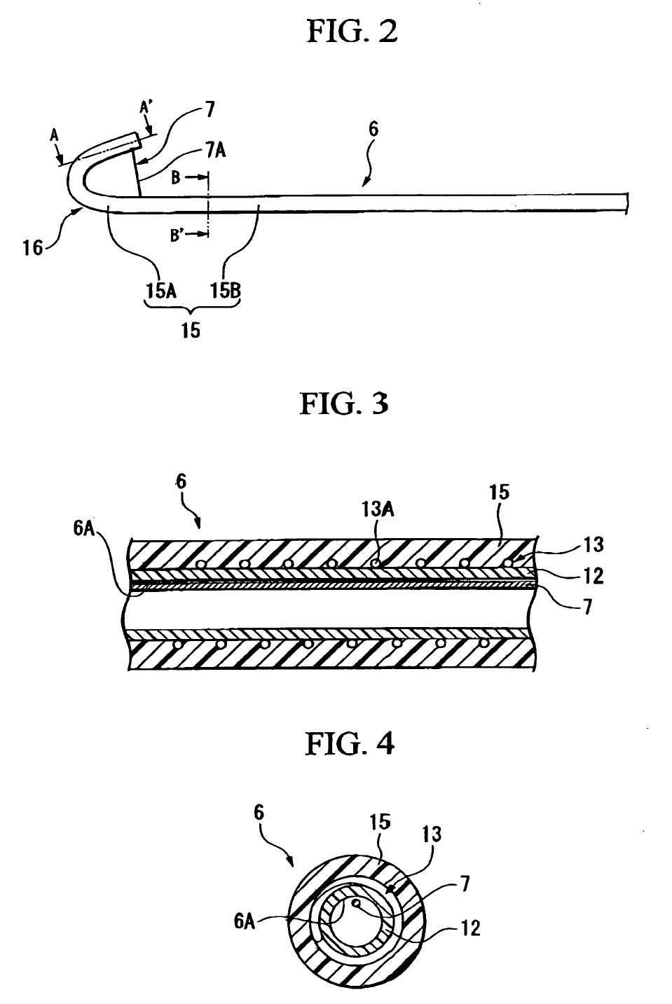

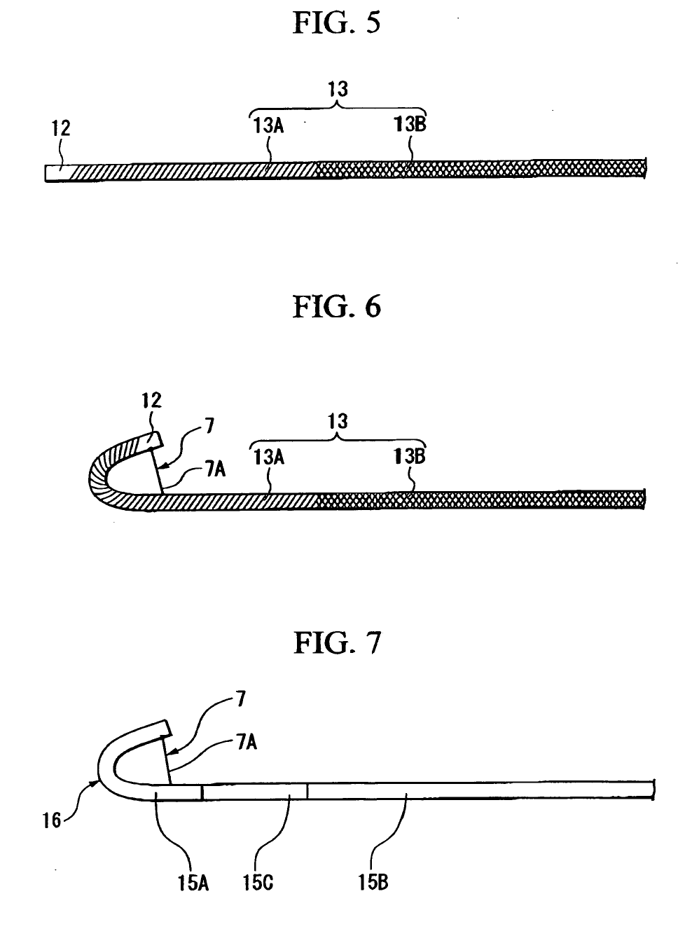

As shown in FIG. 2 to FIG. 6, the curvable catheter 6 includes: a first resin layer 12 serving as an innermost layer made from a fluororesin such as PTFE, PFA, FEP, and ETFE; a metal layer 13 arranged outside of the first resin layer 12; and a second resin layer 15 as the outermost layer arranged further outside of the metal layer 13. The metal layer 13 includes: a coil layer 13A made from a stainless steel or the like arranged on the distal side; and a mesh pipe 13B continually provided on the proximal end of the coil layer 13A. The element wire of the coil layer 13A and the element wire of the mesh pipe 13A are made from the same members. The element wires of the coil layer and the mesh pipe may be respectively made from separate members. The coil layer 13A is formed from a coiled a flat plate or round cross-section wire rod, and for example the flat plate has a length of 0.3 mm and a thickness of 0.08 mm, and the distance of a gap between the flat plate is 0.1 mm to 0.5 mm, and preferably 0.2 mm and 0.3 mm.

The second resin layer 15 includes a relatively soft distal layer 15A arranged on the distal side of the curvable catheter 6; and a relatively hard proximal layer 15B arranged on the proximal side. As shown in FIG. 7, between the distal layer 15A and the proximal layer 15B may be arranged an intermediate layer 15C having a flexibility intermediate between them. The second resin layer 15 is made from a resin such as polyurethane and nylon, and is further separated into the abovementioned respective layers according to the difference in the hardness of the same resin.

The distal layer 15A is arranged in a region including the exposed portion 7A. Moreover, the region arranged with the distal layer 15A becomes a curvable portion 16 which is curved by change of the length of the exposed portion 7A of the operation wire 7 according to the amount that the operation wire 7 is moved to the proximal side of the curvable catheter 6 by the operation portion 8. The portion arranged with the proximal layer 15B is more rigid than the distal side, and thus superior in the torque transmission property. The curvable portion 16 is curvable within a range between 0 to 170 degree of a curved angle, assuming that the curved angle is an angle defined by the axial centers of the proximal end and the distal end, and the curved angle becomes 0 when the axial centers of the proximal end and the distal end are approximately on the same line having the curvable portion 16 therebetween.

The inner diameter of the curvable catheter 6 is 1.5 mm to 5.7 mm, preferably 2.0 mm to 4.5 mm, and more preferably 2.6 mm to 3.3 mm. Moreover, the wall thickness of the curvable catheter 6 is 0.05 mm to 0.5 mm, and preferably 0.1 mm to 0.4 mm. This is based on an assumption that the contrast medium injecting catheter 2 having a minimum outer diameter of 1.3 mm can be inserted therein and the curvable catheter 6 is inserted into the channel 5 of the endoscope 3 having an inner diameter of 6.0 mm.

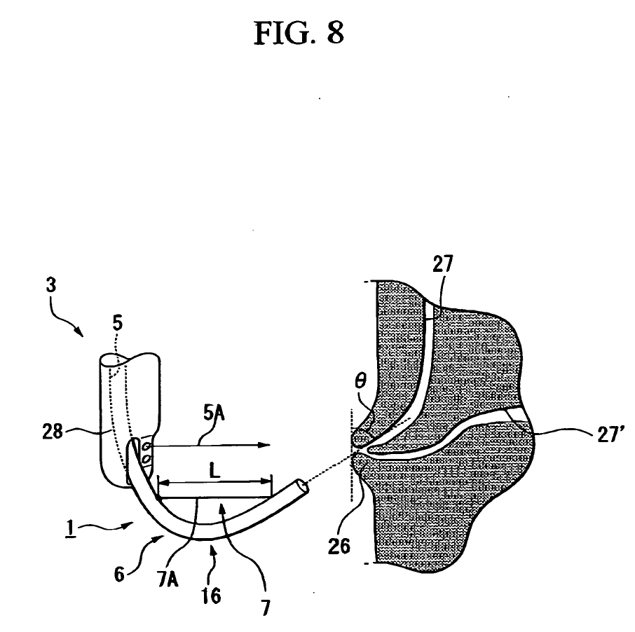

As shown in FIG. 8, the distal end of the operation wire 7 is connected to the vicinity of the distal end of the curvable catheter 6. The length L of the exposed portion 7A varies within a range more than 10 mm but less than 40 mm, depending on an angle θ defined by the distal direction of the curvable catheter 6 and the proximal direction of the curvable catheter 6 by curving the curvable portion 16.

The operation portion 8 includes an operation portion mainbody 17 which is detachably connected to the branched portion 11 and extended in the axial direction, and a slider 18 which is connected with the proximal end of the operation wire 7 and is relatively movable with respect to the operation portion mainbody 17. The travel distance of the slider 18 with respect to the operation portion mainbody 17 has a sufficient length for the angle θ to be changed from 10 degrees to 90 degrees.

The operation portion 8 is provided with a ratchet mechanism (not shown), enabling to move the slider 18 only to the proximal side of the operation portion mainbody 17. By pressing a release button 20 provided on the slider 18, the slider 18 can be also moved to the distal side of the operation portion mainbody 17.

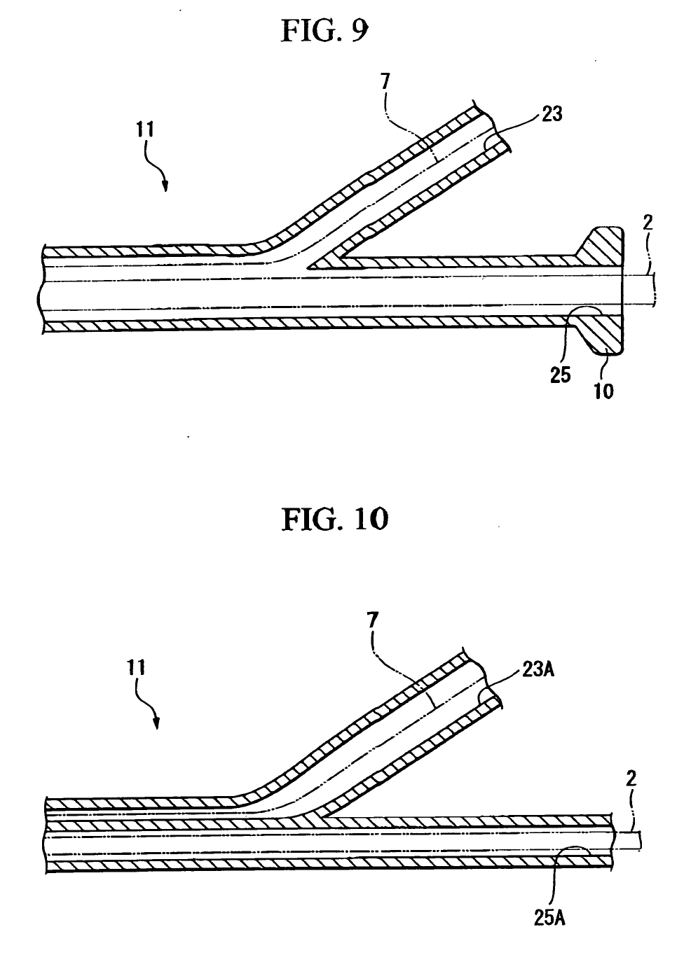

The branched portion 11 includes a first connector 21 which is detachably connected to the proximal end of the curvable catheter 6, and a second connector 22 which is detachably connected to the operation portion mainbody 17. Moreover, as shown in FIG. 9, the branched portion 11 is provided with: a first through hole 23 which is communicated with the through hole 6A, and is inserted with the operation wire 7 arranged in the curvable catheter 6; and a second through hole 25 which is communicated with the through hole 6A, and inserted with an endoscope treatment tool such as the contrast medium injecting catheter 2, branched in the middle. As shown in FIG. 10, a first through hole 23A and a second through hole 25A may be respectively and separately extended to the distal end of the curvable catheter.

Next is a description of the effect of the treatment tool inserting/withdrawing auxiliary device 1 according to the present embodiment, together with a medical procedure through an endoscope using this. As the following medical procedure, the description is regarding a manipulation such as inserting the contrast medium injecting catheter 2 into a duodenal papilla 26 using the endoscope 3, injecting a contrast medium into a bile duct 27 to diagnose under X-ray fluoroscopy, and removing all bile duct calculi. For a manipulation regarding the pancreatic duct 27′, in principle, the bile duct 27 is replaced with a pancreatic duct 27′ in the following description.

Firstly, the insertion portion 28 of the endoscope 3 is inserted into the mouth of a patient (not shown), and the distal end of the insertion portion 28 is positioned in the vicinity of the duodenal papilla 26 through the esophagus (not shown). Then, by performing an angling operation or twisting operation of the endoscope 3, the line of sight 5A is adjusted so that the duodenal papilla 26 can be kept within the endoscope image. Next, the abovementioned curvable catheter 6 of the treatment tool inserting/withdrawing auxiliary device 1 is inserted from the forcep port (not shown) of the endoscope 3 into the channel 5, and made to project from the channel 5. At this time, the slider 18 is moved with respect to the operation portion mainbody 17 so that the curvable catheter 6 smoothly projects.

Next, the operator (not shown) grasps and moves the slider 18 of the operation portion 8 backward with respect to the operation portion mainbody 17 while observing the distal end of the curvable catheter 6 in an observation image. At this time, since the proximal side of the operation wire 7 is moved to the proximal side with respect to the curvable catheter 6, the length of the exposed portion 7A is shortened according to the pulled amount thereof. According to the length of this exposed portion 7A, the curvable portion 16 is curved and the distal end of the curvable catheter 6 is moved to the proximal direction of the curvable catheter 6.

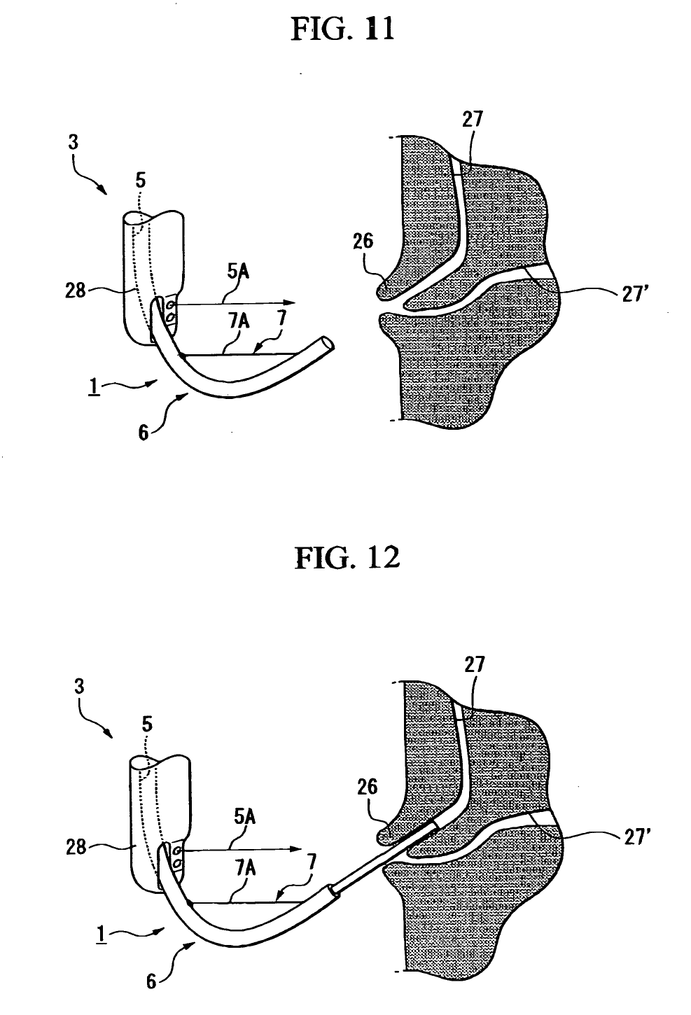

When the slider 18 is moved to a predetermined position with respect to the operation portion mainbody 17, the slider 18 is fixed by the ratchet mechanism (not shown), and the curved angle of the curvable portion 16 is fixed in a condition where it is curved at a predetermined angle within a range between 90 degrees to 170 degrees that is suitable for inserting into the papilla. Furthermore, the operator grasps the branched portion 11 and rotates the curvable catheter 6 with respect to the channel 5, so as to match the distal direction of the curvable catheter 6 with the direction of the bile duct 27 as shown in FIG. 11. The operation of the slider 18 of the operation portion 8, and the rotation operation and the forward moving operation of the curvable catheter 6 may be performed not only by the operator but also by an assistant.

After the curvable catheter 6 is positioned, the contrast medium injecting catheter 2 is inserted from the treatment tool insertion/withdrawal port 10 through the second through hole 25 into the through hole 6A of the curvable catheter 6. The contrast medium injecting catheter 2 may be previously inserted into the curvable catheter 6. Moreover, as shown in FIG. 12, the contrast medium injecting catheter 2 is made to project from the distal end of the curvable catheter 6, and inserted to a predetermined position in the bile duct 27. Then, the contrast medium is poured into the contrast medium injecting catheter 2, and the inside of the bile duct 27 is visually observed by means of X-ray contrast radiography.

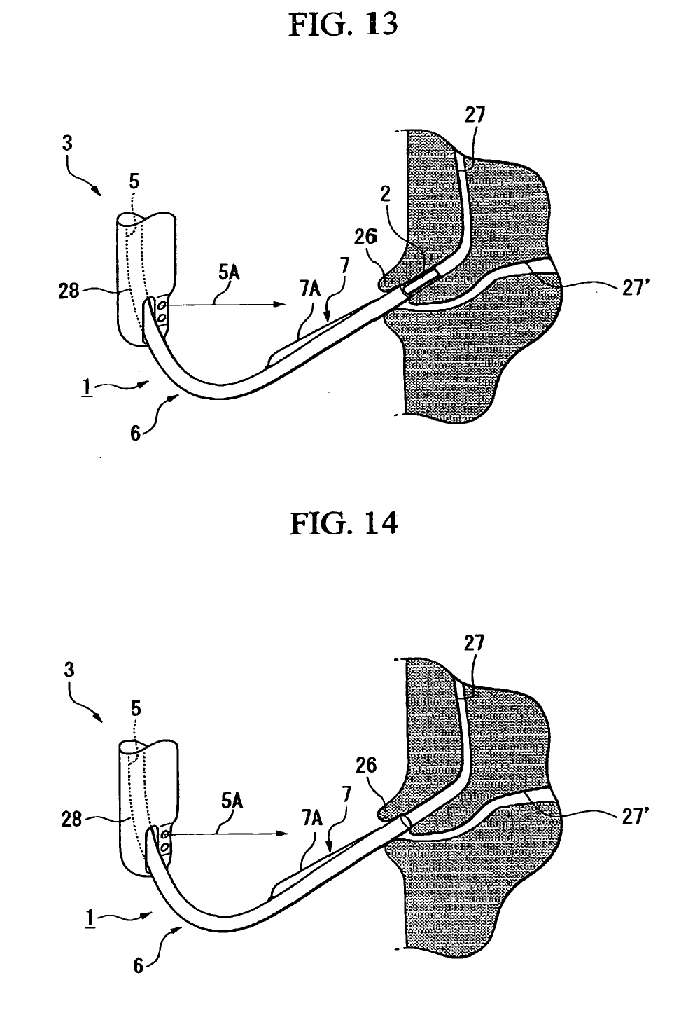

In this condition, the release button 20 is pushed to bring the slider 18 into a slidable condition with respect to the operation portion mainbody 17, and the curvable catheter 6 is pushed out from the channel 5. At this time, while canceling the curved condition of the curvable portion 16, as shown in FIG. 13, the distal end of the curvable catheter 6 is inserted from the duodenal papilla 26 into the bile duct 27.

Moreover, in a condition where the position of the insertion portion 28 of the endoscope 3 and the position of the curvable catheter 6 are fixed, as shown in FIG. 14, the contrast medium injecting catheter 2 is withdrawn from the treatment tool insertion/withdrawal port 10 of the curvable catheter 6.

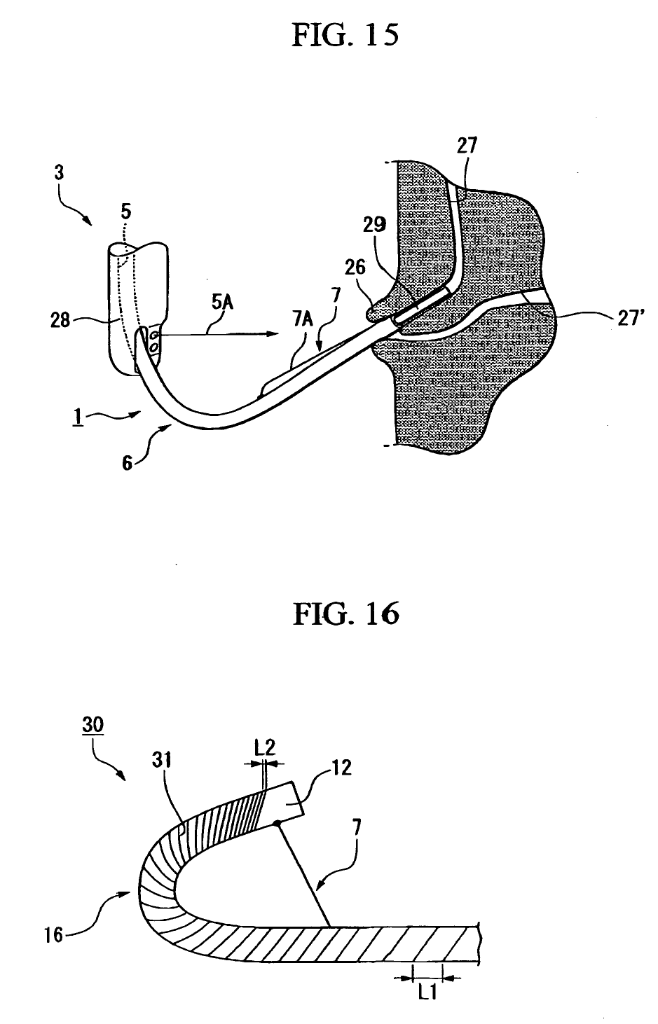

After the withdrawal, the treatment tool insertion/withdrawal port 10 of the curvable catheter 6 is inserted with another treatment tool for an endoscope (second treatment tool) 29 such as a balloon, instead. As shown in FIG. 15, the treatment tool for an endoscope 29 is made to project from the curvable catheter 6 into the bile duct 27, to perform a predetermined treatment on the target site. If another treatment is to be further performed, the treatment tool for an endoscope 29 is withdrawn and another treatment tool for an endoscope (not shown) is inserted.

According to this treatment tool inserting/withdrawing auxiliary device 1 and medical procedure through an endoscope, the distal direction of the curvable catheter 6 projecting from the channel 5, by curving the curvable portion 16 of the curvable catheter 6, may be brought closer to the curvable catheter 6 by a predetermined angle, such as a direction of 10 degree, with respect to the proximal direction of the curvable catheter 6 in the channel 5. Therefore, while the observation image by the endoscope 3 is fixed, the distal direction of the curvable catheter 6 can be matched with the direction of the bile duct 27 by merely curving the curvable portion 16 of the curvable catheter 6. As a result, without requiring a high skill for positioning, cannulation can be readily performed in a condition where the distal end of the endoscope insertion portion 28 is fixed with respect to the duodenal papilla 26.

Moreover, the treatment tool is not directly inserted into the channel 5, but inserted into the curvable catheter 6 which has been previously inserted therein. Therefore, for exchanging the treatment tool, the curvable catheter 6 can be used as a guide, and the treatment tool can be readily exchanged without requiring the guide wire.

Furthermore, the distal side of the second resin layer 15 of the curvable catheter 6 is a soft distal layer 15A, and the curvable portion 16 is arranged with the coil layer 13A. Therefore, when the curvable portion 16 is curved, a large curved amount can be obtained without buckling the curvable catheter 6. On the other hand, the proximal side of the second resin layer 15 is a hard proximal layer 15B, and is arranged with the mesh pipe 13B. Therefore, while maintaining a predetermined rigidity, insertion/withdrawal into/from the channel 5 can be readily performed, and the rotation torque when rotated with respect to the channel 5 can be suitably transferred to the distal side.

Moreover, the first resin layer 12 serving as the innermost layer of the curvable catheter 6 contains a fluororesin. Therefore the frictional force can be reduced, and the contrast medium injecting catheter 2 and another endoscope treatment tool inserted into the through hole 6A can be smoothly inserted/withdrawn. The first resin layer may contain a hydrophilic resin.

Moreover, in the example of inserting the contrast medium injecting catheter 2, the effect of the treatment tool inserting/withdrawing auxiliary device 1 was described. However, another treatment tool for an endoscope 29 such as a balloon may be inserted instead of the contrast medium injecting catheter 2 at the beginning.

### Second Embodiment

A second embodiment of the present invention is described with reference to the drawings. The difference between the second embodiment and the first embodiment is the point that, as shown in FIG. 16, a treatment tool inserting/withdrawing auxiliary device 30 according to the present embodiment has a pitch L1 on the proximal side of the coil layer 31 greater than a pitch L2 on the distal side thereof.

The pitch L2 in the vicinity of the curvable portion 16 has the same interval as that of the coil layer 13A according to the first embodiment. The pitch L1 in the connection part with the mesh pipe 13B is 0.5 mm to 0.6 mm, and the pitch in the middle is changed so that the pitch gradually becomes greater from the distal side to the proximal side.

According to this treatment tool inserting/withdrawing auxiliary device 30 and medical procedure through an endoscope, a similar effect to that of the first embodiment can be demonstrated. Moreover, by using this device, a similar medical procedure can be performed. In particular, since the pitch of the coil layer 31 is small on the distal side in the vicinity of the curvable portion 16, the curvable portion 16 can be curved without buckling. Moreover, since the pitch is gradually changed, in the connection part between the coil layer 31 and the mesh pipe 13B, discontinuous change with respect to the curve rigidity can be made less than that of the first embodiment, and the buckling resistance can be improved.

### Third Embodiment

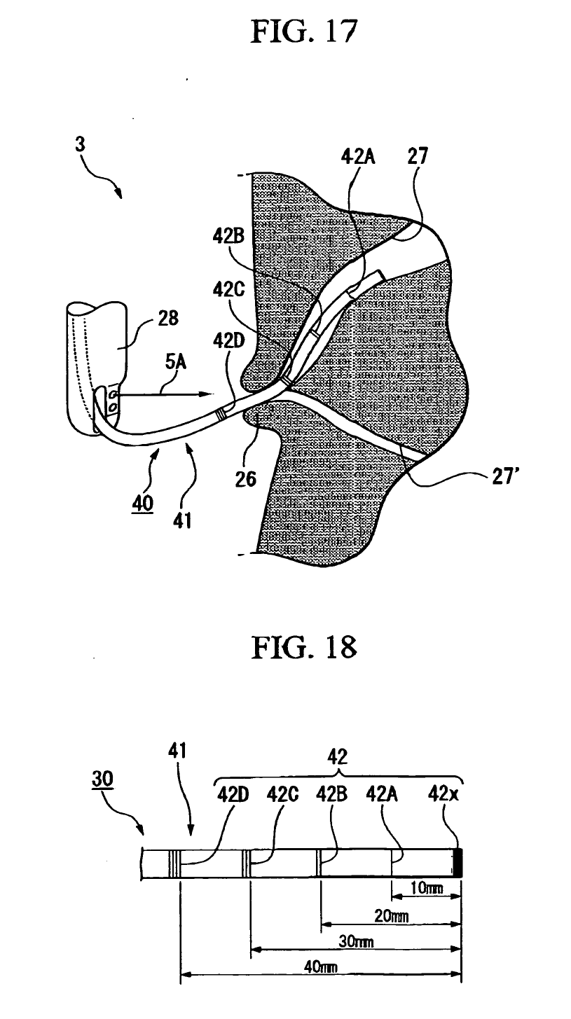

A third embodiment of the present invention is described with reference to the drawings. The difference between the third embodiment and the first embodiment is the point that, as shown in FIG. 17 and FIG. 18, the distal end of a curvable catheter 41 of a treatment tool inserting/withdrawing auxiliary device 40 according to the present embodiment is provided with visual check markers 42 for identifying the length from the distal end.

The visual check markers 42 include for example: a reference visual check marker 42X provided at the most distal end of the curvable catheter 41; a first position 42A provided in a position 10 mm from the distal end of the curvable catheter 41; a second position 42B provided in a position 20 mm therefrom; a third position 42C provided in a position 30 mm therefrom; and a fourth position 42D provided in a position 40 mm therefrom. The respective positions may be provided by an X-ray impermeable material so as to be observable under X-ray contrast radiography. Moreover, the number of the provided markers and the distance from the distal end of the curvable catheter 41 are not limited to the above, and may be provided according to the manipulation.

According to this treatment tool inserting/withdrawing auxiliary device 40 and medical procedure through an endoscope, since the visual check markers 42 are provided, it can be ascertained in an X-ray image under X-ray contrast radiography how far the distal side of the curvable catheter 41 is inserted into the bile duct and the like. Moreover, even if not under X-ray contrast radiography, the visual check markers 42 can be confirmed by an endoscopic image.

### Fourth Embodiment

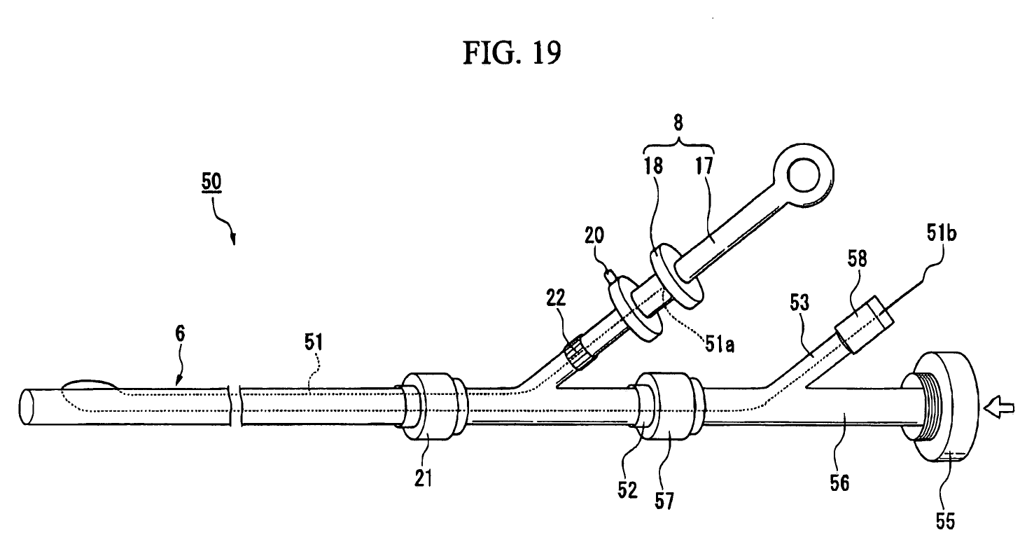

A fourth embodiment of the present invention is described with reference to the drawings. The difference between the fourth embodiment and the first embodiment is the point that, as shown in FIG. 19, an operation wire 51 of a treatment tool inserting/withdrawing auxiliary device 50 according to the present embodiment has one end 51a connected to the operation portion mainbody 17 of the operation portion 8, and the other end 51b side folded at the distal end of the curvable catheter 6 and arranged toward the proximal side.

To a treatment tool insertion/withdrawal port 52 of the branched portion 11 is detachably connected an extension portion 56 that is provided with a new treatment tool insertion port 55 and a wire insertion/withdrawal port 53 through which the other end 51b side of the operation wire 51 is inserted, via a connector 57. While the extension portion 56 is connected, the second through hole 25 of the branched portion 11, the wire insertion/withdrawal port 53, and the new treatment tool insertion/withdrawal port 55 are communicated.

The operation wire 51 projecting from the wire insertion/withdrawal port 53 is arranged with a clasp 58 for preventing the operation wire 51 from being pulled into the wire insertion/withdrawal port 53. This clasp 58 is formed larger than the inner diameter of the wire insertion/withdrawal port 53, and detachably attached to the operation wire 51.

Next is a description of the effect of the treatment tool inserting/withdrawing auxiliary device 50 according to the present embodiment, together with a medical procedure through an endoscope using this.

First, similarly to the first embodiment, the curvable catheter 6 of the treatment tool inserting/withdrawing auxiliary device 50 is made to project from the distal opening of the channel of an endoscope (not shown).

Next, an operator (not shown) performs a similar operation to that of the first embodiment while observing an observation image. In a condition where the curvable portion 16 is curved at a predetermined angle to match the distal direction of the curvable catheter 6 with the direction of the bile duct (not shown), the contrast medium injecting catheter (not shown) is inserted into the bile duct. Then, the contrast medium is poured into the contrast medium injecting catheter, and the inside of the bile duct is visually observed by means of X-ray contrast radiography.

When the curvable catheter 6 is inserted into the bile duct, the clasp 58 is taken off from the operation wire 51, and further the connector 22 is separated to take out the operation portion 8 from the curvable catheter 6. At this time, the other end 51b side of the operation wire 51 is moved to the distal direction in the curvable catheter 6, is folded at the distal end, and goes again toward the proximal side, to be pulled out from the curvable catheter 6 together with the operation portion 8.

The curvable catheter 6 is inserted into the bile duct, and after withdrawing the operation wire 51 and the operation portion 8, the contrast medium injecting catheter 2 is withdrawn from the treatment tool insertion/withdrawal port 55 of the curvable catheter 6. The treatment tool insertion/withdrawal port 55 of the curvable catheter 6 is inserted with another treatment tool for an endoscope such as a balloon (not shown), instead, to perform a predetermined treatment on the target site.

According to this treatment tool inserting/withdrawing auxiliary device 50 and medical procedure through an endoscope, the operation wire 51 can be withdrawn from the curvable catheter 6 after the contrast medium injecting catheter is inserted into the bile duct. Therefore, when the endoscope treatment tool is inserted into the curvable catheter 6, the treatment tool can be more readily exchanged without being interfered with by the operation wire 51.

### Fifth Embodiment

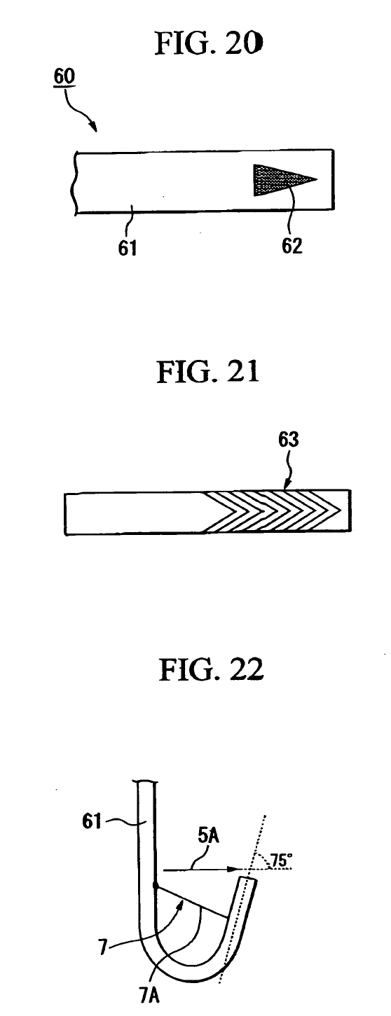

A fifth embodiment of the present invention is described with reference to the drawings. The difference between the fifth embodiment and the first embodiment is the point that, as shown in FIG. 20, the distal outer face of a curvable catheter 61 of a treatment tool inserting/withdrawing auxiliary device 60 according to the present embodiment is provided with an index 62 formed to be gradually wider toward the proximal side.

The index 62 is formed in an approximate isosceles triangle, and is arranged in a predetermined position on the distal side from the exposed portion 7A of the operation wire 7, so that the apex of the isosceles faces to the distal side of the curvable catheter 61. The shape of the index 62 is not limited to an approximate isosceles triangle. As shown in FIG. 21, there may be an index 63 where a plurality of only the isosceles portions of isosceles triangles are arranged in a row in the longitudinal direction of the curvable catheter 61.

Next is a description of the effect of the treatment tool inserting/withdrawing auxiliary device 60 according to the present embodiment, together with a medical procedure through an endoscope using this.

First, similarly to the first embodiment, the distal end of the insertion portion of an endoscope (not shown) is positioned in the vicinity of the duodenal papilla 26, to capture the duodenal papilla 26 in the endoscope image. Next, the curvable catheter 61 of the treatment tool inserting/withdrawing auxiliary device 60 is inserted into the channel, so as to project from the distal opening of the channel.

An operator (not shown) performs a similar operation to that of the first embodiment while observing an observation image V by means of the endoscope, to curve the curvable portion 16 at a predetermined angle, so that the distal end of the curvable catheter 61 faces toward the proximal direction of the curvable catheter 61.

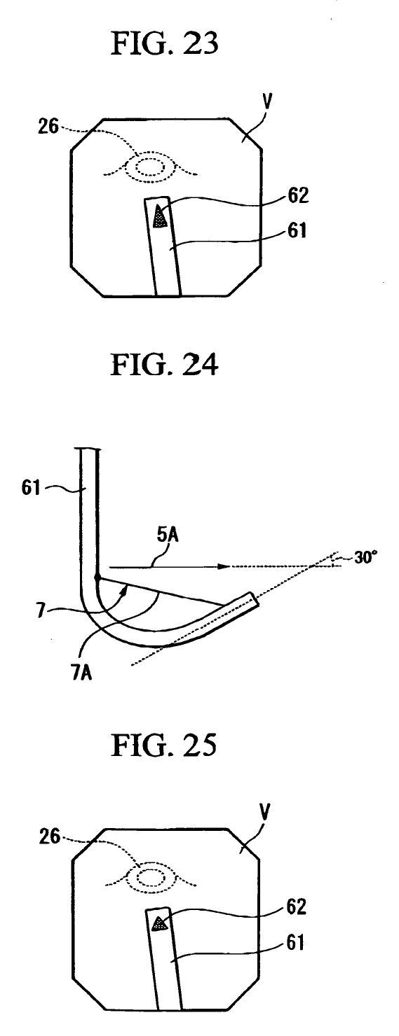

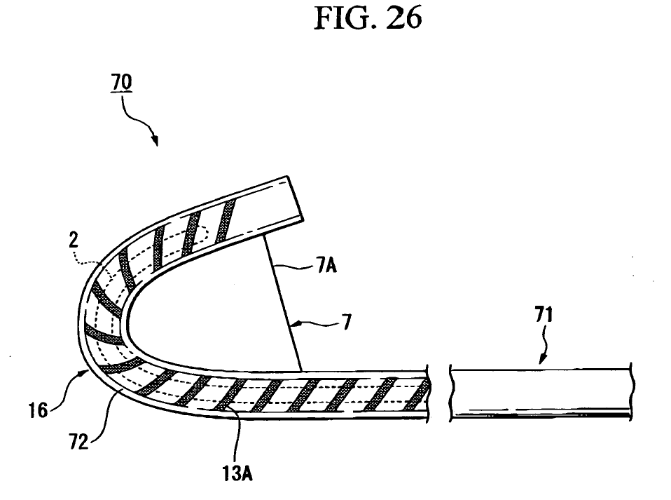

At this time, if the distal end of the curvable catheter 61 is arranged in a direction approximately orthogonal to the line of sight 5A (75 degree direction in the drawing) as shown in FIG. 22, the index 62 appears in a nearly original form of the approximate isosceles triangle in the observation image as shown in FIG. 23. On the other hand, if the distal end of the curvable catheter 61 is arranged close to the direction of the line of sight 5A (30 degree direction in the drawing) as shown in FIG. 24, the index 62 appears in a squashed form in the height direction in the observation image as shown in FIG. 25.

Here, in order to insert a contrast medium injecting catheter (not shown) into the bile duct 27, it is required that the distal end of the curvable catheter 61 faces the direction of 11 o'clock with respect to the duodenal papilla 26. In order to insert it into the pancreatic duct (not shown), it is required that the distal end of the curvable catheter 61 faces the direction of 4 o'clock with respect to the duodenal papilla 26. Therefore, the shape of the index 62 is observed to judge from the shape in the image whether or not the distal direction of the curvable catheter 61 is matched with the direction of the bile duct 27 or the pancreatic duct 27′. Then the contrast medium injecting catheter that has been inserted into the curvable catheter 61 is pushed out, and the distal end of the contrast medium injecting catheter is inserted from the duodenal papilla 26 into the bile duct 27.

According to this treatment tool inserting/withdrawing auxiliary device 60 and medical procedure through an endoscope, the shape of the index 62 in the observation image appears differently according to the curved angle of the distal end of the curvable catheter 61 with respect to the insertion portion 28. Therefore, the operator can readily judge whether or not the distal end of the curvable catheter 61 faces toward a predetermined direction by visually checking the shape of the index 62 in the observation image.

### Sixth Embodiment

A sixth embodiment of the present invention is described with reference to the drawings. The difference between the sixth embodiment and the first embodiment is the point that, as shown in FIG. 26, a first resin layer (not shown) and a second resin layer 72 in the region including the curvable portion 16 of a curvable catheter 71 of a treatment tool inserting/withdrawing auxiliary device 70 according to the present embodiment are made from a transparent resin.

The first resin layer and the second resin layer 72 are respectively made from resins such as nylon, urethane, PTFE, and PFA. As a result, the position of the distal end of a treatment tool such as the contrast medium injecting catheter 2 passing through the inside of the curvable catheter 71 can be visually checked in an observation image of the endoscope.

According to this treatment tool inserting/withdrawing auxiliary device 70 and medical procedure through an endoscope, in the region including the transparent resin, a treatment tool passing through the inside can be visually checked in an observation image of the endoscope, and the position of the treatment tool with respect to the curvable catheter 71 can be readily determined.

### Seventh Embodiment

A seventh embodiment of the present invention is described with reference to the drawings. The difference between the seventh embodiment and the first embodiment is the point that the surface of the proximal side of a curvable catheter 81 of a treatment tool inserting/withdrawing auxiliary device 80 according to the present embodiment is provided with convex portions 82.

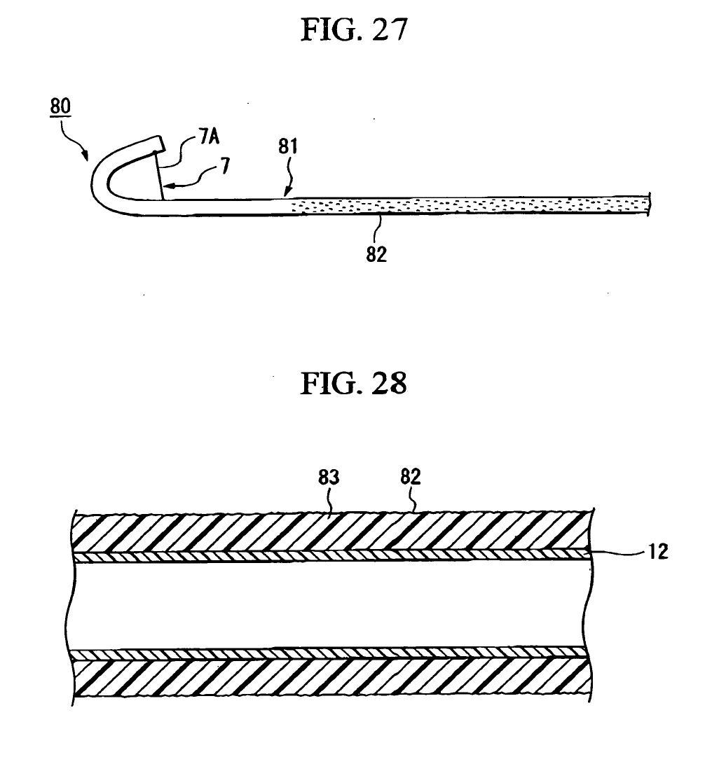

The convex portions 82 are minute and are formed on the second resin layer 83 on the proximal side from the curvable portion 16 as shown in FIG. 27 and FIG. 28. The convex portions 82 may be provided not only on the proximal side of the curvable catheter, but also to the distal end. Moreover, concavities may be provided instead of the convex portions 82.

According to this treatment tool inserting/withdrawing auxiliary device 80 and medical procedure through an endoscope, because the surface of the curvable catheter 81 is provided with the convex portions 82, when the curvable catheter 81 is inserted into a channel (not shown), the frictional force with the wall surface of the channel can be reduced and the curvable catheter 81 can be readily inserted/withdrawn. Moreover, instead of the convex portions 82, or in addition to the convex portions 82, the surface of at least the proximal side of the curvable catheter 81 may be a hydrophilic lubricant surface.

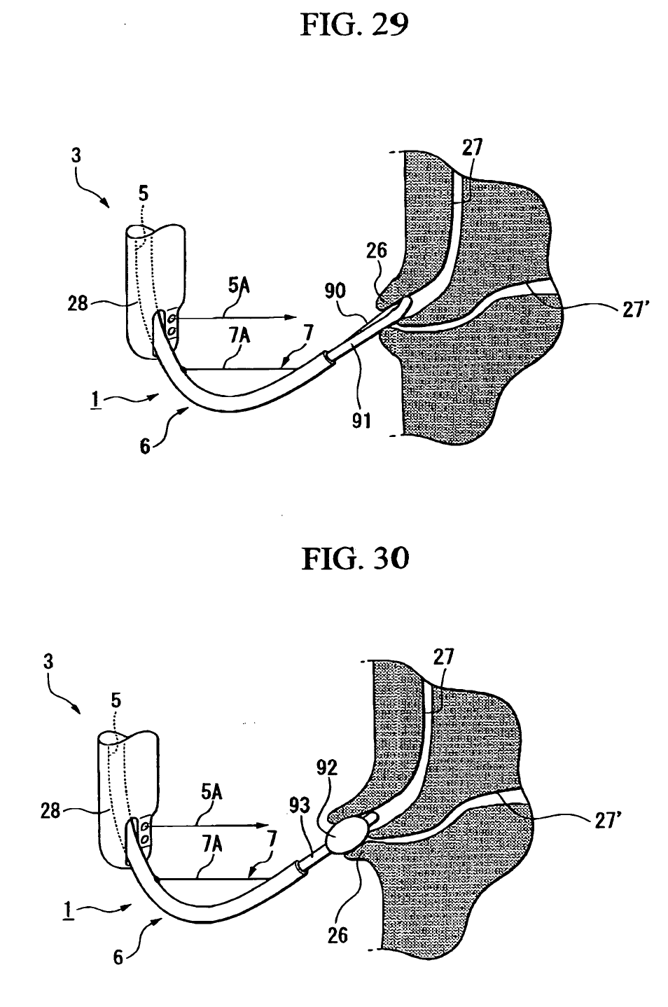

The technical scope of the present invention is not limited to the above embodiments, and various modifications can be made without departing from the scope of the present invention. For example, there may be used a contrast medium injecting catheter 91 provided with an incision knife 90 connected to a high frequency power source (not shown) on the distal end as shown in FIG. 29, or a contrast medium injecting catheter 93 provided with a balloon 92 as shown in FIG. 30.

In this case, in the abovementioned medical procedure, the contrast medium is poured into the contrast medium injecting catheter 91 or 93, and the inside of the bile duct 27 is visually observed by means of X-ray contrast radiography. Then, without withdrawing the contrast medium injecting catheter 91 or 93 from the treatment tool insertion/withdrawal port (not shown) of the curvable catheter 6, the sphincter of the duodenal papilla 26 can be incised as it is by the incision knife 90, or the sphincter of the duodenal papilla 26 can be extended by the balloon 92.

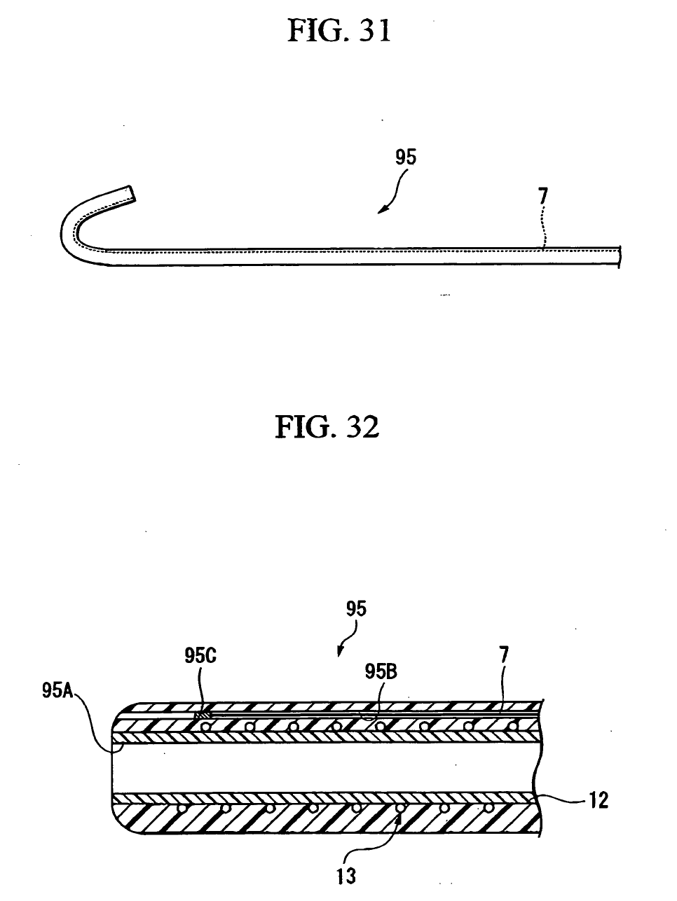

Moreover, as shown in FIG. 31 and FIG. 32, while the operation wire 7 without an exposed portion 7A is inserted into a through hole 95B for a wire which is provided in the curvable catheter 95 separately from the through hole 95A, the distal end thereof may be fixed to a fixing portion 95C provided in the vicinity of the distal end of the curvable catheter 7 (about 0.5 mm to 5.0 mm from the distal end). Here, the reason for being in the vicinity of the distal end is that the distal end of the curvable catheter is tapered. In this case, the curvable catheter 95 can also be curved.

What is claimed is:

1. A treatment tool inserting/withdrawing auxiliary device comprising:
a catheter through which a treatment tool for an endoscope is to be inserted, and which is insertable into a channel of a flexible endoscope;
an operation wire which is inserted into the catheter, and is supported on a distal end or the vicinity of the catheter;
an operation portion which moves the operation wire back and forth with respect to the catheter; and
a curvable portion which is provided on the catheter and is curved by a back and forth movement of the operation wire, wherein
the curvable portion is curvable within a range between 0 to 170 degree of a curved angle, assuming that the curved angle is an angle defined by axial centers of the proximal end and the distal end of the curvable portion, and the curved angle becomes 0 when the axial centers of the proximal end and the distal end are approximately on a same line having the curvable portion therebetween.

2. The treatment tool inserting/withdrawing auxiliary device according to claim 1, wherein the operation wire is partially exposed to the outside of the catheter to form an exposed portion, a length of the exposed portion varies according to a moved amount of the operation wire to the proximal side of the catheter by the operation portion, and the curvable portion is curved according to the length of the exposed portion.

3. The treatment tool inserting/withdrawing auxiliary device according to claim 1, wherein a coil layer is arranged at least on the curvable portion of the catheter.

4. The treatment tool inserting/withdrawing auxiliary device according to claim 3, wherein a mesh pipe layer is continually provided on the proximal end of the coil layer, and arranged on the catheter.

5. The treatment tool inserting/withdrawing auxiliary device according to claim 3, wherein a fluororesin layer is arranged at least on the inside of the coil layer of the catheter.

6. The treatment tool inserting/withdrawing auxiliary device according to claim 3, wherein a pitch on the proximal side of the coil layer is greater than a pitch of the distal side thereof.

7. The treatment tool inserting/withdrawing auxiliary device according to claim 1, wherein a visual check marker for identifying a length from the distal end is provided on the distal end of the catheter.

8. The treatment tool inserting/withdrawing auxiliary device according to claim 1, comprising a treatment tool insertion/withdrawal port which is provided in communication with the catheter, and through which the treatment tool for an endoscope is to be inserted/withdrawn.

9. The treatment tool inserting/withdrawing auxiliary device according to claim 8, wherein one end of the operation wire is connected to the operation portion, and the other end side is folded on the distal end of the catheter and arranged toward the proximal side of the catheter.

10. The treatment tool inserting/withdrawing auxiliary device according to claim 9, wherein the operation portion is detachably connected to the catheter.

11. The treatment tool inserting/withdrawing auxiliary device according to claim 1, wherein a length of the exposed portion varies within a range of more than 10 mm and less than 40 mm, by a back-and-forth moving operation of the operation portion.

12. The treatment tool inserting/withdrawing auxiliary device according to claim 1, wherein the distal end of the catheter is provided with an index at least a part of which is formed to be gradually wider toward the proximal side.

13. The treatment tool inserting/withdrawing auxiliary device according to claim 1, wherein at least the curvable portion of the catheter comprises a transparent resin.

14. The treatment tool inserting/withdrawing auxiliary device according to claim 1, wherein a surface of the proximal side of the catheter is provided with convex portions or concavities.

15. A medical procedure through an endoscope comprising:
inserting a distal end of a catheter of a treatment tool inserting/withdrawing auxiliary device, a distal direction of which is curved at a predetermined angle with respect to a proximal direction, through a channel of a flexible endoscope, to arrange in the vicinity of a duodenal papilla;
matching the distal direction of the catheter with the direction of a bile duct or a pancreatic duct, while the distal end of the endoscope is fixed;
inserting a first treatment tool into the catheter; and
making the first treatment tool project from the catheter, to be inserted into the bile duct or the pancreatic duct.

16. A medical procedure through an endoscope comprising:
inserting a distal end of a catheter of a treatment tool inserting/withdrawing auxiliary device, a distal direction of which is curved at a predetermined angle with respect to a proximal direction, through a channel of a flexible endoscope, to arrange in the vicinity of a duodenal papilla;
matching the distal direction of the catheter with the direction of a bile duct or a pancreatic duct, while the distal end of the endoscope is fixed;
inserting a first treatment tool into the catheter;
making the first treatment tool project from the catheter, to be inserted into the bile duct or the pancreatic duct;
inserting the catheter into the bile duct or the pancreatic duct using the first treatment tool as a guide;
withdrawing the first treatment tool from the catheter; and
inserting a second treatment tool differing from the first treatment tool into the catheter, to perform a treatment.

17. The medical procedure through an endoscope according to one of claims 15 and 16, wherein the catheter is provided with an operation wire, and the distal side of the catheter is curved by pulling the operation wire.

18. The medical procedure through an endoscope according to one of claims 15 and 16, wherein the catheter has a torque transmission property.

19. The medical procedure through an endoscope according to one of claims 15 and 16, wherein the distal side of the first treatment tool is further provided with an incision wire capable of incising a duodenal papilla sphincter.

20. The medical procedure through an endoscope according to one of claims 15 and 16, wherein the distal side of the first treatment tool is further provided with a balloon capable of extending a duodenal papilla sphincter.
